(19) United States
(12) Patent Application Publication
(10) Pub. No.: US 2024/0188871 A1
(43) Pub. Date: Jun. 13, 2024

(54) ELASTIC STIMULATING DILATOR CLIP FOR NEUROMONITORING

(71) Applicant: RHYTHMLINK INTERNATIONAL, LLC, COLUMBIA, SC (US)

(72) Inventor: Brian PETREE, West Columbia, SC (US)

(21) Appl. No.: 18/538,278

(22) Filed: Dec. 13, 2023

Related U.S. Application Data
(60) Provisional application No. 63/432,157, filed on Dec. 13, 2022.

(51) Int. Cl. A61B 5/254; A61B 5/296

(52) U.S. Cl. CPC A61B 5/254; A61B 5/296; A61B 2560/0468

(57) ABSTRACT

Disclosed are various embodiments of a dilator clip which may include a first member and a second member attached at a central hinged and aligned, wherein a first member handle and a second member handle are adapted for compressing to move the dilator clip into an open state. The dilator clip may further comprise a compression band in which compressive forces are applied to secure the dilator clip against a dilator. The dilator clip may further comprise a first or second member with an embedded electrode for monitoring electrical signals. Further disclosure provides methods for utilizing and acquiring electrical/neurological signals from the dilator clip.

### CROSS-REFERENCE TO RELATED APPLICATIONS

The present invention relates to and claims priority to U.S. Provisional Patent Application No. 63/432,157, filed on Dec. 13, 2022, entitled Elastic Stimulating Dilator Clip for Neuromonitoring, the contents of which are hereby incorporated in the entirety.

### FIELD

The present invention relates to neurological monitoring, in particular to systems and methods relating to the use of dilators in continuous real-time electromyography (EMG).

### BACKGROUND

The use and practice of minimally invasive surgeries to minimize patient complications and morbidity is on the rise. With the miniaturization of equipment, including advancements in optical apparatuses such as laparoscopes, practitioners are able to perform complex surgeries without requiring large incisions. Lumbar interbody fusion is one particular procedure that has continued to make advancements under a new procedure known as extreme lateral interbody fusion (XLIF).

XLIF differs from traditional spinal surgeries as the practitioner accesses the space between each spinal disc from the patient's side, rather than from the front or back, thus avoiding contact with major back muscles, bones, and ligaments. XLIF includes accessing the spine through placement of a series of tubular dilators with incrementally increasing diameters as well as the use of a retractor device. Continuous, real-time EMG is required to monitor the patient's status during an XLIF procedure. EMG is also called a myogram, and it measures the muscle response or electrical activity in response to a nerve's stimulation of the muscle. An EMG is utilized during a procedure to detect neuromuscular abnormalities and complications with an XLIF, or to assure the practitioner the procedure is progressing accordingly.

In performing an XLIF procedure a practitioner utilizes tubular dilators to ensure the nerves of the lumbar plexus remain unharmed, and that the practitioner has safe access through the psoas to the spine. An electrical connection between the various dilators and EMG equipment is required throughout the procedure to deliver and record stimulus for neuromonitoring. This connection is vital, and the ability to secure such a connection while the procedure is ongoing is paramount to obtaining accurate readings.

In most procedures, a conductive clip is attached to an uncoated, conductive member of the dilator to provide an electrical connection for neurological monitoring. One issue that may arise is the integrity of the electrical connection between the clip and the multiple dilators of varying diameters. The clip may provide a tight, and in some cases difficult, fit around the largest diameter dilator while providing a loose connection to the smallest diameter dilator potentially leading to intermittent or loss of electrical connection.

In some existing dilator clips, the members are manually operated, requiring the user to apply force to bring the pressure regions of the clip together. This manual operation can be cumbersome and may result in inconsistent pressure being applied to the dilator, potentially affecting the accuracy of the monitoring as it relates to contact made by an electrode. Additionally, these clips often lack an embedded electrode, requiring separate electrode placement on the dilator, which can be time-consuming and may introduce additional sources of error.

Other approaches have attempted to address these issues by incorporating mechanical force elements into the hinge of the dilator clip. These force elements are designed to automatically apply a consistent mechanical force to drive the pressure regions of the clip together, ensuring a secure and stable grip on the dilator. However, these previous designs require a different size of dilator clip per the size of the dilator, which further increases medical supply costs.

In summary, there is a need in XLIF and other procedures that require a dilator clip to create a consistent electrical connection to a wide range of dilators, each of which may comprise a different diameter. Furthermore, there is a need to produce low cost and effective solutions that do not require multiple sizing dimensions, and in which can rapidly be applied to existing operating room procedures.

### SUMMARY

In some aspects, the techniques described herein relate to a dilator clip for neurological monitoring during continuous real-time electromyography, including: a first member and a second member, including a first member handle region, a second member handle region, a first member pressure region, and second member pressure region; a central hinge connecting the first member and the second member, the central hinge including a mechanical force element that applies mechanical force to drive the first member pressure region and the second member pressure region together; a compression band secured through an opening on the first member pressure region and the second member pressure region; and an embedded electrode within the second member pressure region, wherein the embedded electrode is configured to contact a tubular dilator.

In some aspects, the techniques described herein relate to a dilator clip, wherein the compression band is included of an elastic band.

In some aspects, the techniques described herein relate to a dilator clip, further including a housing structure within the second member with a cavity for housing a lead wire to the embedded electrode.

In some aspects, the techniques described herein relate to a dilator clip, further including the lead wire soldered to the embedded electrode.

In some aspects, the techniques described herein relate to a dilator clip, wherein the compression band further includes conductive material to acquire and/or deliver electrical signals.

In some aspects, the techniques described herein relate to a dilator clip, wherein the central hinge is included of a pin that integrates with a torsion spring that forces the first member pressure region and the second member pressure region into a closed state by applying pressure to the first member pressure region and the second member pressure region.

In some aspects, the techniques described herein relate to a dilator clip, wherein the compression band is coated with a layer of lubricant to have a low friction coefficient to allow for rotation around a central axis of the tubular dilator.

In some aspects, the techniques described herein relate to a dilator clip, wherein the dilator clip is included of a semi-rigid polymeric material.

In some aspects, the techniques described herein relate to a dilator clip, further including a plurality of compression bands.

In some aspects, the techniques described herein relate to a dilator clip, wherein the central hinge is included of a compression spring.

In some aspects, the techniques described herein relate to a method of acquiring signals from a dilator clip, including: providing a first member and a second member, including a first member handle region, a second member handle region, a first member pressure region, and second member pressure region, and a central hinge connecting the first member and the second member, the central hinge including a mechanical force element that applies mechanical force to drive the first member pressure region and the second member pressure region together, and a compression band secured through an opening on the first member pressure region and the second member pressure region, and an embedded electrode within the second member pressure region, wherein the embedded electrode is configured to contact a tubular dilator; applying pinching pressure to the first member handle region and the second member handle region causing the central hinge to move the first member pressure region and the second member pressure region to an open state; placing the first member pressure region and the second member pressure region around the tubular dilator so that the compression band is in contact with the tubular dilator; and releasing pressure on the first member handle region and the second member handle region causing the central hinge to decompress and force the first member pressure region and the second member pressure region into a closed state.

In some aspects, the techniques described herein relate to a method, further including acquiring electrical signals through the embedded electrode.

In some aspects, the techniques described herein relate to a method, further including providing a housing structure within the second member with a cavity for housing a lead wire to the embedded electrode.

In some aspects, the techniques described herein relate to a method, further including soldering a lead wire to the embedded electrode.

In some aspects, the techniques described herein relate to a method, further including providing the compression band with a conductive material to acquire and/or deliver electrical signals.

In some aspects, the techniques described herein relate to a method, further including providing a pin on the central hinge that integrates with a torsion spring that forces the first member pressure region and the second member pressure region into the closed state.

In some aspects, the techniques described herein relate to a method, further including providing the compression band with a lubricant coating to have a low friction coefficient to allow for rotation around a central axis of the tubular dilator.

In some aspects, the techniques described herein relate to a method, further including providing the dilator clip including a semi-rigid polymeric material.

In some aspects, the techniques described herein relate to a method, further including providing a plurality of compression bands for attaching to the first member and the second member.

In some aspects, the techniques described herein relate to a method, further including removing the dilator clip by compressing the first member handle region and the second member handle region and moving the first member pressure region and the second member pressure region away from the tubular dilator.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. In the drawings:

FIG. 1A is an illustration of an example dilator clip in a closed state;

FIG. 1B is an illustration of the example dilator clip in 1A in an open state;

FIG. 1C is an illustration of the example dilator clip of 1A in a closed state as applied to a tubular dilator;

FIG. 2A is an illustration of an example dilator clip as applied to a small diameter tubular dilator; and

FIG. 2B is an illustration of the example dilator clip of 2B as applied to a large diameter tubular dilator.

FIG. 3A is an illustration of an example dilator clip as applied to a small diameter tubular dilator;

FIG. 3B is an illustration of the example dilator clip of 3B as applied to a large diameter tubular dilator; and

FIG. 4 is a flow diagram of an example method of applying a dilator clip to a tubular dilator.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### I. Applications and Methods of Use

In one aspect, a dilator clip is configured with a compression band comprised of elastic that provides tensioning around a range of diameters of tubular dilators. Said aspect provides for a single dilator clip that may be used across a range of dilators, without requiring additional dilator clips. The compression bands are typically attached through openings in the first and second member, wherein excess compression band may be stored to allow for a range of tubular dilators. Thus simplifying the equipment needed for performing accurate neuromonitoring. Furthermore, the dilator clip may have an integrated tensioning central hinge as well as an embedded electrode integrated within the dilator clip.

In another aspect, the elastic material of the dilator clip provides constant pressure across a range of dilator diameters, thus providing constant contact with an electrode embedded into the body of the dilator clip housing. In another aspect, the elastic material is itself conductive and may act as the electrode providing stimulus to the dilators or receiving signals therefrom. In said aspect the elastic material may be impregnated with metallic additives, or may otherwise be comprised of a conductive plastic such as a polymer comprised from carbon, a resin, or other additive that allows for the transfer of electrical signals. In another aspect the body of the elastic stimulating dilator clip may be comprised of acrylonitrile butadiene styrene (ABS), or other semi-rigid polymeric material that provides insulating properties to the embedded electrode (e.g. PET, PETG, Polypropylene, Polyethylene, Polyvinyl Chloride, Polycarbonate), and wherein the compression band may have an electrically conductive coating such as mixture of silver and silver chloride, and may be impregnated with a conductive material such as copper fibers or carbon fibers, such as graphite fibers.

In another aspect, the elastic material is coated in a lubricant or additives added to have a slippery, low friction surface, to allow rotation of the clip around the axis of the tubular dilator while maintaining constant connection between the dilator and embedded electrode. Such a coating may also be used as a bacterial preventative agent, or as an agent to hold the integrity of the elastic material. Furthermore, such a coating may also impart conductive behavior and allow for the transfer of electrical signals for EMG monitoring.

### II. With Reference to the Drawings

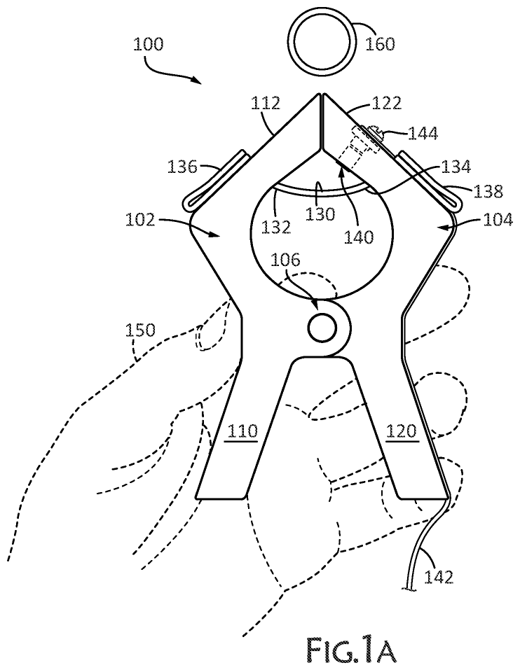

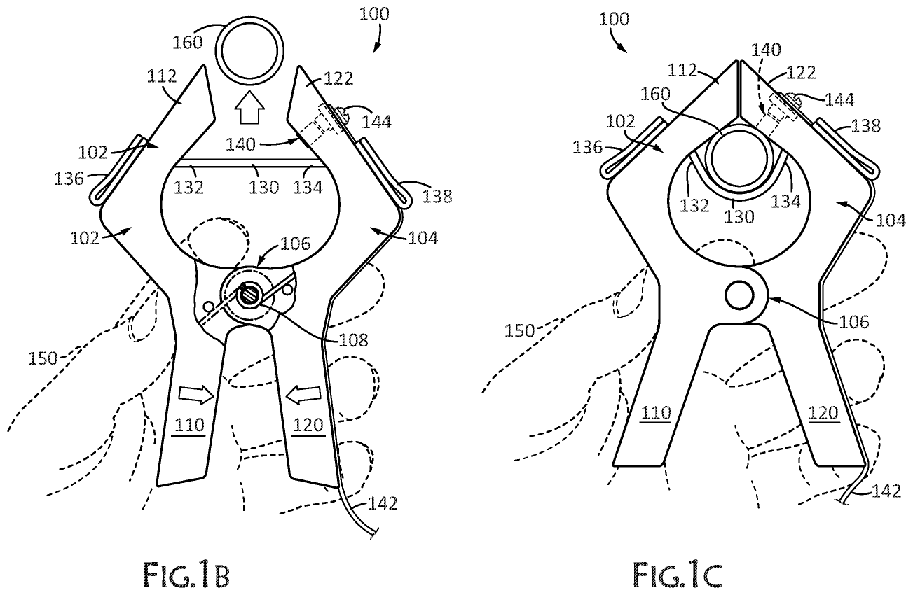

Referring now to FIG. 1, an example of an elastic stimulating dilator clip 100, commonly referred to as a dilator clip, for neurological monitoring. In the example, a first member 102 and a second member 104 are disclosed, wherein the members comprise a first member handle region 110, a second member handle region 120, a first member pressure region 112, and second member pressure region 122. Each member, having a handle region and a pressure region, wherein the handle region is designed to be handled by a practitioner 150 to allow pinching pressure to retract the pressure region into an open state for placement on a tubular dilator 160. The pressure region, by default, is in a closed state, due to the central hinge exerting force to close the dilator clip. In a closed state the pressure regions on the first and second member are in contact, or near contact. The pressure region contains the embedded electrode 140, that may be secured on by a fastener 144. In other embodiments, the embedded electrode 140 is entirely internal to the first or second member, with only the surface of the embedded electrode 140 visible and prepared to make electrical contact with the tubular dilator 160.

Continuing, in FIG. 1, a central hinge 106 is equipped to secure and connect the first and second member, as well as to create force to drive the pressure regions on the first and second member into a closed state. In one aspect, the central hinge 106 comprises a mechanical force element that applies mechanical force to drive the first member pressure region 112 and the second member pressure region 122 together to clasp around a tubular dilator and to be held securely by a compression band 130. In further aspects, the central hinge 106 is comprised of a pin that integrates with a torsion spring that forces the first member pressure region 112 and the second member pressure region 122 into a closed state by applying pressure to the first member pressure region 112 and the second member pressure region 122.

The compression band 130, in one aspect, is secured through an opening on the first member pressure region 132 and an opening on the second member pressure region 134. Excess compression band 130 may be stored externally through a wrapping on the housing (136, 138) that allows the compression band 130 to accommodate a range of tubular dilators. In other aspects the compression band may be integrated within the housing of the first member and the second member's pressure region. The compression band 130 allows for adding resistance and pressure to a tubular dilator of various sizes. In one aspect, the compression band 130 is comprised of an elastic or rubber material, and may decompress and compress based on physical force to enable it to securely hold a tubular dilator of various sizes. In this aspect, the compression band allows the disclosure herein to be replicated across various tubular dilators, without the need for additional dilator clip sizes.

Continuing, an embedded electrode 140 is located within a pressure region and situated to make contact with a tubular dilator 160. The embedded electrode may be comprised of conductive material, and depending upon the preferences of the monitoring equipment, contain graphite, or noble metals, or even copper, titanium, or brass, so long as the properties remain of transporting electrical signals.

In one aspect, the elastic stimulating dilator clip 100 may have a housing structure within the first or second member that comprises a cavity for housing a lead wire 142 to the embedded electrode 140. The housing structure (not depicted) would allow routing of the lead wire 142 internal to the member upon which side the embedded electrode resides, allowing for cable management and less risk of disconnecting the lead wire from the embedded electrode. In further aspects, the lead wire is soldered to the embedded electrode, and may further be wrapped with an insulating wrap. In other aspects, the lead wire may be exterior to the housing and adhered to the side of the left or right member, through gluing, taping, forming, molding, or otherwise integrating along the member so as to avoid disconnecting the lead wire from the embedded electrode.

Continuing, in further aspects, the compression band 130 may further comprise a conductive material to acquire and/or deliver electrical signals. In this aspect, the embedded electrode 140 becomes the compression band 130, and thus the compression band 130 itself serves as the electrode. In this aspect, the lead wire 142 would be configured to the compression band 130, and the lead wire 142 would be directed to monitoring equipment for sending and receiving electrical signals from the tubular dilator 160. In even further aspects, the compression band 130 may be coated with a layer of lubricant to have a low friction coefficient to allow for rotation and movement around a central axis of the tubular dilator 160. In this instance, the compression band 130 may need adjusting along the tubular dilator 160, and to provides the means to do so a lubricant may be included as an additive to the compression band 130, for example silicon lubricants or other sterile lubricants. Furthermore, the compression band may have its strength increased by adding a plurality of compression bands, or otherwise increasing the size and scale of the compression band. Typical compression bands have a round profile, however, flat profiles are also contemplated. Furthermore, a tubular dilator 160 may also be any shape or form of dilator, the purpose being to provide access to a cavity within the body and to allow for monitoring of electrical signals and the passage of instruments.

Referring now to FIG. 1B, an example of the dilator clip 100 of FIG. 1A in an open state. In this aspect the practitioner 150 is pressing on the first member handle region 110 and the second member handle 120 to decompress the central hinge 108, comprising a torsion spring, that allows the first member pressure region 112 and the second member pressure region 122 to retract to an open state. In an open state the dilator clip can pass over and clip onto the tubular dilator 160. When doing so the compression band 130 is stretched as the dilator clip 100 is transitioned into an open state.

An open state increases tension on the compression band 130, and ensures the compression band is in a taught or tensioned state to receive, hold, and place pressure on the tubular dilator so as to reduce movement and to hold the embedded electrode 140 securely against the tubular dilator 160.

In FIG. 1B, the central hinge 108 is disclosed as a torsion spring, this torsion spring is placed against pins on the first and second member that allow the members to be held in a closed state unless acted upon by a practitioner 150. In other aspects, the central hinge 108 may be an extension spring, a coil spring, a tensioned hinge, or other mechanical embodiment that allows for continuous pressure on each member to hold it in a closed state, but also capable of retracting to an open state.

Continuing, in FIG. 1C, the dilator clip 100 of FIGS. 1A-B is disclosed now deployed on a tubular dilator 160, wherein the compression band 130 is engaged against the tubular dilator 160 to securely hold the embedded electrode 140 against the tubular dilator 160 for sending and receiving electrical signals. In this aspect, the dilator clip 100 is returned to a closed state by the central hinge 106 acting upon the members, as well as the compression band 130 securing the tubular dilator 160.

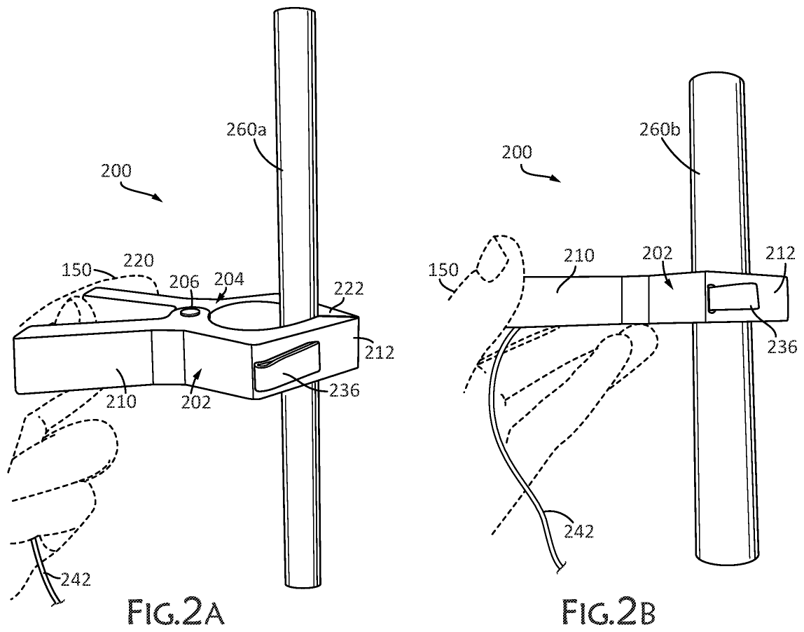

In FIG. 2A, an example dilator clip 200 is disclosed securely configured with a small diameter tubular dilator 260a. This aspect discloses the versatility of a single dilator clip being capable of configuring with a range of diameters of dilators during procedures such as XLIF. The practitioner 150 is positioning the dilator clip 200 to securely engage the compression band 236, which is disclosed as having the ability to store excess compression band material to accommodate a range of tubular dilators. In this aspect, the first member handle region 220 and the second member handle region 210 are engaged by the practitioner 150 in orienting the dilator clip 200 around the small diameter tubular dilator 260a.

FIG. 2B disclosed an example dilator clip 200 of FIG. 2A, securely configuring around a large diameter tubular dilator 260b. This aspect discloses the versatility of a single dilator clip being capable of configuring with a range of diameters of dilators during procedures such as XLIF. The practitioner 150 is positioning the dilator clip 200 to securely engage the compression band 236, which is disclosed as having the ability to release excess band material to accommodate a larger tubular dilator. In this aspect, the first member handle region 220 and the second member handle region 210 are engaged by the practitioner 150 in orienting the dilator clip 200 around the large diameter tubular dilator 260b.

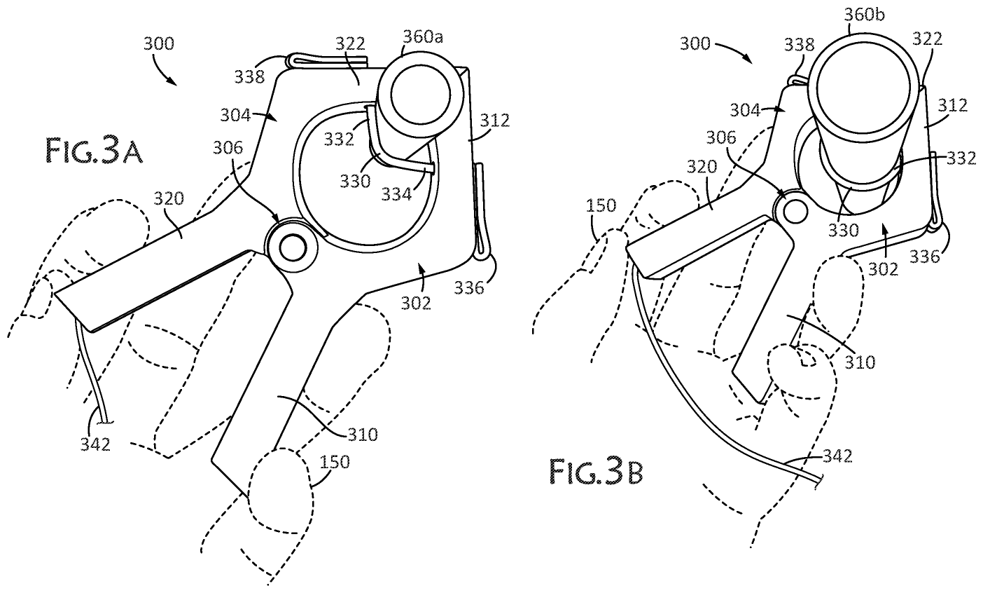

FIGS. 3A-B disclose additional perspective views of FIGS. 2A-B, disclosing a single dilator clip 300 as being equipped to configure with a range of tubular dilators (360a, 360b). Similar to previous aspects, a practitioner may engage a first member handle region 320 and a second member handle region 310 to move the dilator clip 300 into an open and a closed state by acting upon a central hinge 306. This action provides mechanical force to open the first member pressure region 304 and the second member pressure region 302 to allow clamping or securing around a tubular dilator. In the aspect of FIG. 3A, a small tubular dilator 360a is disclosed, wherein the compression band (338, 336) is cinched or otherwise secured with compressive forces and tensioning against the small tubular dilator 360a. In the aspect of FIG. 3B, a large tubular dilator 360b is disclosed, wherein the compression band (338, 336) is extend around the diameter of the larger tubular diameter to hold the embedded electrode in place.

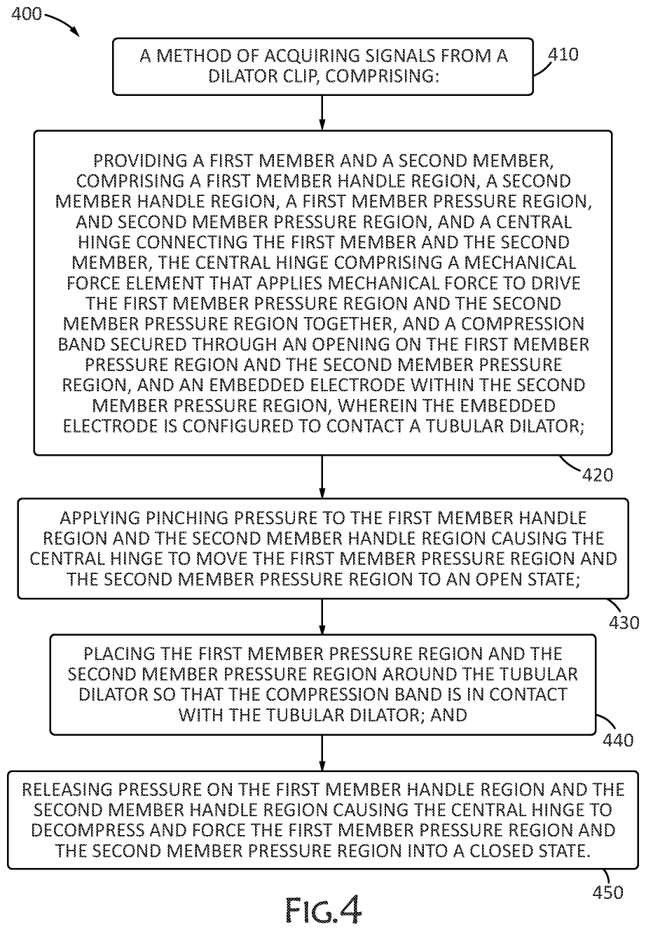

Referring now to FIG. 4, an example method of acquiring signals from a dilator clip 400. First, providing a first member and a second member, comprising a first member handle region, a second member handle region, a first member pressure region, and second member pressure region, and a central hinge connecting the first member and the second member, the central hinge comprising a mechanical force element that applies mechanical force to drive the first member pressure region and the second member pressure region together, and a compression band secured through an opening on the first member pressure region and the second member pressure region, and an embedded electrode within the second member pressure region, wherein the embedded electrode is configured to contact a tubular dilator 410. Next, applying pinching pressure to the first member handle region and the second member handle region causing the central hinge to move the first member pressure region and the second member pressure region to an open state 420. Next, placing the first member pressure region and the second member pressure region around the tubular dilator so that the compression band is in contact with the tubular dilator 430. Lastly, releasing pressure on the first member handle region and the second member handle region causing the central hinge to decompress and force the first member pressure region and the second member pressure region into a closed state 440. In additional steps, the method further comprises acquiring electrical signals through the embedded electrode by contacting the tubular dilator. In other aspects, the method may send electrical signals to the tubular dilator from monitoring/transmitting equipment through the lead wire to the embedded electrode.

Continuing with FIG. 4, the example method further comprises applying an elastic stimulating dilator clip around a tubular dilator for XLIF procedures. The method comprises steps to apply and acquire neurological signals utilizing an elastic stimulating dilator clip, also known as a dilator clip. In one aspect, the method comprises providing an elastic stimulating dilator clip with a first member, a second member and a central hinge comprising a torsion spring, wherein the first member comprises a first member handle, a first member pressure region with an opening for securing a first end of a compression band, and wherein the second member comprises a second member handle, a second member pressure region, an embedded electrode, and an opening for securing a second end of the compression band.

Next, the method comprises applying pressure to the first member handle and the second member handle causing the torsion spring to compress and move the first member pressure region and the second member pressure region to an open state. Next, the method comprises placing the first and second members around a tubular dilator so that the compression band and the embedded electrode is in contact with the tubular dilator. Next, releasing pressure on the first and second members handle regions causing the torsion spring to compress and force the first and second member to a closed state. Lastly, acquiring neurological signals through the embedded electrode on either a first or second member.

It should be emphasized that the above-described embodiments of the present disclosure are merely possible examples of implementations set forth for a clear understanding of the principles of the disclosure. Many variations and modifications may be made to the above-described embodiments without departing substantially from the scope and principles of the disclosure. All such modifications and variations are intended to be included herein within the scope of this disclosure and protected by the following claims.

Therefore, the following is claimed:

1. A dilator clip for neurological monitoring during continuous real-time electromyography, comprising:
a first member and a second member, comprising a first member handle region, a second member handle region, a first member pressure region, and second member pressure region;
a central hinge connecting the first member and the second member, the central hinge comprising a mechanical force element that applies mechanical force to drive the first member pressure region and the second member pressure region together;
a compression band secured through an opening on the first member pressure region and the second member pressure region; and
an embedded electrode within the second member pressure region, wherein the embedded electrode is configured to contact a tubular dilator.

2. The dilator clip of claim 1, wherein the compression band is comprised of an elastic band.

3. The dilator clip of claim 1, further comprising a housing structure within the second member with a cavity for housing a lead wire to the embedded electrode.

4. The dilator clip of claim 3, further comprising the lead wire soldered to the embedded electrode.

5. The dilator clip of claim 1, wherein the compression band further comprises conductive material to acquire and/or deliver electrical signals.

6. The dilator clip of claim 1, wherein the central hinge is comprised of a pin that integrates with a torsion spring that forces the first member pressure region and the second member pressure region into a closed state by applying pressure to the first member pressure region and the second member pressure region.

7. The dilator clip of claim 1, wherein the compression band is coated with a layer of lubricant to have a low friction coefficient to allow for rotation around a central axis of the tubular dilator.

8. The dilator clip of claim 1, wherein the dilator clip is comprised of a semi-rigid polymeric material.

9. The dilator clip of claim 1, further comprising a plurality of compression bands.

10. The dilator clip of claim 1, wherein the central hinge is comprised of a compression spring.

11. A method of acquiring signals from a dilator clip, comprising:
providing a first member and a second member, comprising a first member handle region, a second member handle region, a first member pressure region, and second member pressure region, and a central hinge connecting the first member and the second member, the central hinge comprising a mechanical force element that applies mechanical force to drive the first member pressure region and the second member pressure region together, and a compression band secured through an opening on the first member pressure region and the second member pressure region, and an embedded electrode within the second member pressure region, wherein the embedded electrode is configured to contact a tubular dilator;
applying pinching pressure to the first member handle region and the second member handle region causing the central hinge to move the first member pressure region and the second member pressure region to an open state;
placing the first member pressure region and the second member pressure region around the tubular dilator so that the compression band is in contact with the tubular dilator; and
releasing pressure on the first member handle region and the second member handle region causing the central hinge to decompress and force the first member pressure region and the second member pressure region into a closed state.

12. The method of claim 11, further comprising acquiring electrical signals through the embedded electrode.

13. The method of claim 11, further comprising providing a housing structure within the second member with a cavity for housing a lead wire to the embedded electrode.

14. The method of claim 11, further comprising soldering a lead wire to the embedded electrode.

15. The method of claim 11, further comprising providing the compression band with a conductive material to acquire and/or deliver electrical signals.

16. The method of claim 11, further comprising providing a pin on the central hinge that integrates with a torsion spring that forces the first member pressure region and the second member pressure region into the closed state.

17. The method of claim 11, further comprising providing the compression band with a lubricant coating to have a low friction coefficient to allow for rotation around a central axis of the tubular dilator.

18. The method of claim 11, further comprising providing the dilator clip comprising a semi-rigid polymeric material.

19. The method of claim 11, further comprising providing a plurality of compression bands for attaching to the first member and the second member.

20. The method of claim 11, further comprising removing the dilator clip by compressing the first member handle region and the second member handle region and moving the first member pressure region and the second member pressure region away from the tubular dilator.
